(19) United States
(12) Patent Application Publication
(10) Pub. No.: US 2002/0098154 A1
(43) Pub. Date: Jul. 25, 2002

(54) Methods and apparatus for medicating the nasal sinuses

(76) Inventor: Gordon Wayne Dyer, Northwood, NH (US)

Correspondence Address: Gordon Wayne Dyer, 12 Murray Lane, Northwood, NH, 03261, US

(21) Appl. No.: 09/765,894

(22) Filed: Jan. 20, 2001

(51) Int. Cl. A61L 9/04; A61K 9/14; A61K 31/56

(52) U.S. Cl. 424/45; 514/179; 424/46; 514/254.07

(57) ABSTRACT

The present invention provides a method and accompanying apparatus for supplying medications, particularly antibiotics, to the deeper parts areas of the sinuses. The pressure of application from use of the Valsalva maneuver and the use of medications that are both water and fat-soluble aids the medications in penetrating deep into the sinuses. When the medication is an antibiotic, this has the benefit of delivering a high level of antibiosis using a line of antibiotics that the likely bacteria will not be as resistant to because they have not had as much prior exposure to this antibiotic. The lighter-than-air propellant aids in delivering the medication to those sinus areas superior to the nose. If the infection extends to the eardrums, making the Valsalva maneuver painful, or if the patient is simply unusually sensitive, then earplugs to reduce the stress on the eardrums may be worn while the patient performs the Valsalva maneuver.

### BACKGROUND OF THE INVENTION

1. Field of the Invention

The present invention relates to methods for applying sinus medications. More particularly, the present invention provides methods for topically treating sinus allergies and sinus infections.

2. Description of Related Art

In recent years the standard medical treatment for chronic allergic sinusitis has been topical steroid nasal sprays and oral decongestants/anti-histamines. These systemic decongestants and anti-histamines are used to provide weaker, but more penetrating, treatment to the more remote sinus areas that the steroid nasal sprays currently do not reach. In milder chronic allergic sinusitis cases, topical steroid nasal sprays or systemic oral anti-histamines may be used separately to control the allergic sinusitis. Also in milder chronic allergic sinusitis cases, mast cell stabilizer nasal sprays may be substituted for steroid nasal sprays, with or without concomitant use of systemic oral antihistamines.

In recent years the standard medical treatment for sinus infections has been systemic antibiotics, coupled with concomitant use of systemic nasal decongestants. In more severe sinus infections, particularly if there is an accompanying allergic condition, the systemic antibiotic and decongestant therapy may be augmented with topical steroid nasal sprays and oral decongestant/anti-histamines. Topical antibiotic therapy regimens have been proposed in the past, but without any apparent practical utility. However, some recent studies have again been studying the use of topical antibiotic therapy regimens. One of these was given FDA approval in October, 2000. Also, a patent was issued in June, 2000 for administering medications via nasal inhalation using a nebulizer (U.S. Pat. No. 6,076,520).

Sinus allergies are a major medical problem in the United States. Millions of dollars are spent every year on both prescription and other-the-counter sinus allergy, congestion and pain medications. Because the allergy's inherent sinus congestion leads to a warm, moist environment with poor drainage, sinus allergies often lead to sinus infections. Both sinus congestion and sinus infection lead to intense pain and thus additional millions of dollars are spent controlling this pain.

A shortcoming of the present, standard oral regimen for sinus allergies is that chronic use of decongestants, anti-histamines, and analgesics can, respectively, raise blood pressure, cause drowsiness, and cause liver and/or kidney damage. All of these shortcomings also apply to the present, standard oral regimen for treating sinus infections. In addition, due to the recurrent nature of sinus infections and the high antibiotic dosages necessary to treat them, oral regimens for treating sinus infections lead to antibiotic-resistant bacteria.

Oral antibiotic therapies inherently induce antibiotic-resistant bacteria because the antibiotic is introduced not just to the bacteria that are causing the sinus infection, but to all the other endemic bacteria normally present in the body too, such as E. coli and Staph. aureus. This often-repeated-yet-unintended bacterial antibiotic exposure eventually leads to highly antibiotic-resistant bacteria that in turn cause future infections that are difficult to treat. Aggravating this difficulty, the inherent congestion of sinus infections impedes the delivery of the blood borne systemic antibiotic because the congestion impairs the flow of blood to the infected area. Trying to decrease the sinus congestion with steroid sprays, to increase the penetration of the systemic antibiotic, is often unsuccessful because the steroid concomitantly decreases the body's infection fighting ability. Thus the sinus infection worsens in spite of high amounts of powerful systemic antibiotics and often the only recourse is repeated sinus surgery.

In view of the foregoing, it would be desirable to have a way to treat sinus problems topically. An advantage of the current invention is that it provides an alternative route of delivering medications to the sinuses. Another advantage of the current invention is that it provides an alternative group of medications used to treat sinus problems than are currently being used on the public. Another advantage of the current invention is that it provides an alternative, or supplementary, means of treating the sinuses if the oral treatment route currently used by physicians is not sufficiently effective. Another advantage of the current invention is that it provides an alternative use for medications used in treating eye disorders which, due to the natural lacrimal drainage system, routinely end up in the sinuses, anyhow.

### SUMMARY

In accordance with the present invention, methods and an apparatus are provided for treating sinus problems topically.

The present invention includes a method for applying medications to the sinuses by performing a Valsalva maneuver after the medication has been inhaled.

The present invention includes having the medication be both water-soluble and fat-soluble.

The present invention includes having the medication be applied while the patient is wearing earplugs.

The present invention includes having the medication be selected from the group consisting of chloramphenicol, ciprofloxacin, gentamicin, norfloxacin, ofloxacin, tobramycin, polymyxin B, neomycin, trimethoprim, natamycin, povidone-iodine, diclofenac, ketorolac, flurbiprofen, suprofen, idoxuridine, trifluridine, cidofovir, acyclovir, famciclovir, valacvclovir, cromolyn sodium, ketorolac tromethamine, levocabastine ketotifen, iodoxamide, emedastine, olopatadine, loteprednol etabonate, pemerolast potassium, levofloxacin, amphotericin B, nystatin, miconazole, and ketoconazole.

The present invention includes having the medication be a spray of liquid.

The present invention includes having the medication be a drop of liquid.

The present invention includes having the medication be a powder.

The present invention includes having the medication be an antifungal medication.

The present invention includes having the medication be a mast cell stabilizer.

The present invention includes having the medication be a non-steroidal anti-inflammatory drug.

The present invention includes having the medication be a corticosteroid.

The present invention includes having the medication be an antibiotic.

The present invention also includes having a medication applicator that uses a propellant gas selected from the group consisting of nitrogen gas, helium gas and air.

The present invention includes having the applicator use a medication that is both water-soluble and fat-soluble.

The present invention includes having the applicator use a medication that is selected from the group consisting of chloramphenicol, ciprofloxacin, gentamicin, norfloxacin, ofloxacin, tobramycin, polymyxin B, neomycin, trimethoprim, natamycin, povidone-iodine, diclofenac, ketorolac, flurbiprofen, suprofen, idoxuridine, trifluridine, cidofovir acyclovir, famciclovir, valacvclovir, cromolyn sodium, ketorolac tromethamine, levocabastine ketotifen, iodoxamide, emedastine, olopatadine, loteprednol etabonate, pemerolast potassium, levofloxacin, amphotericin B, nystatin, miconazole, and ketoconazole.

The present invention includes having the applicator use a medication that is an antifungal medication.

The present invention includes having the applicator use a medication that is an antibiotic.

The present invention includes having the applicator use a medication that is a mast cell stabilizer.

The present invention includes having the applicator use a medication that is a corticosteroid.

The present invention includes having the applicator be used while the patient wears at least one earplug in his ear canal.

### DETAILED DESCRIPTION OF THE INVENTION

The present invention generally comprises a method for topically applying medications to the sinuses. The invention includes an applicator, medications that are both water and fat-soluble and the use of the Valsalva maneuver. The applicator may use drops of liquid, a spray of powder, or a spray of liquid. The medications can be antibiotics, antifungal, non-steroidal antiinflammatory drugs (NSAIDS), mast cell stabilizers or corticosteroids.

In a preferred embodiment, a spray of liquid is inhaled through a nostril at least one time then, with the patient's mouth shut and while pinching shut his nostrils, exhaling gently until his Eustachian tubes open (a Valsalva maneuver). The Valsalva maneuver creates a positive pressure in the sinuses. The aerosol medication used is both water and fat-soluble. The aerosol applicator is known in the art and uses either air, helium gas, or nitrogen gas as the propellant.

Valsalva maneuver means the common method used by scuba divers to add air pressure to their sinuses to compensate for the increased pressure on their ear drums as they descend.

The medications used by this invention can include: chloramphenicol, ciprofloxacin, gentamicin, norfloxacin, ofloxacin, tobramycin, polymyxin B, neomycin, trimethoprim, natamycin, povidone-iodine, diclofenac, ketorolac, flurbiprofen, suprofen, idoxuridine, trifluridine, cidofovir, acyclovir, famciclovir, valacvclovir, cromolyn sodium, ketorolac tromethamine, levocabastine ketotifen, iodoxamide, emedastine, olopatadine, loteprednol etabonate, pemerolast potassium, levofloxacin, amphotericin B, nystatin, miconazole, and ketoconazole.

The foregoing description is intended to be illustrative and is not to be taken as limiting. Other variations within the spirit and scope of this invention are possible and will be apparent to those skilled in the art.

I claim:

1. A method for applying medications to a person's sinuses comprising:
applying the medication into nostril while simultaneously inhaling a breath through the nostril; and
at least one time, holding the breath and performing a Valsalva maneuver.

2. The method of claim 1 wherein the medication is both water-soluble and fat-soluble.

3. The method of claim 2 wherein the medication is applied while the person has an earplug in his ear canal.

4. The method of claim 2 wherein the medication is selected from the group consisting of chloramphenicol, ciprofloxacin, gentamicin, norfloxacin, ofloxacin, tobramycin, polymyxin B, neomycin, trimethoprim, natamycin, povidone-iodine, diclofenac, ketorolac, flurbiprofen, suprofen, idoxuridine, trifluridine, cidofovir, acyclovir, famciclovir, valacvclovir, cromolyn sodium, ketorolac tromethamine, levocabastine ketotifen, iodoxamide, emedastine, olopatadine, loteprednol etabonate, pemerolast potassium, levofloxacin, amphotericin B, nystatin, miconazole, and ketoconazole.

5. The method of claim 2 wherein medication is a spray of liquid.

6. The method of claim 2 wherein the medication is a drop of liquid.

7. The method of claim 2 wherein the medication is a powder.

8. The method of claim 2 wherein the medication is an antifungal medication.

9. The method of claim 2 wherein the medication is a mast cell stabilizer.

10. The method of claim 2 wherein the medication is a non-steroidal anti-inflammatory drug.

11. The method of claim 2 wherein the medication is a corticosteroid.

12. The method of claim 2 wherein the medication is an antibiotic.

13. An aerosol sinus medication applicator, the improvement comprising having the propellant gas be selected from the group consisting of nitrogen gas and helium gas and air.

14. The applicator of claim 12 wherein the medication applied is both water-soluble and fat-soluble.

15. The applicator of claim 14 wherein the medication is selected from the group consisting of chloramphenicol, ciprofloxacin, gentamicin, norfloxacin, ofloxacin, tobramycin, polymyxin B, neomycin, trimethoprim, natamycin, povidone-iodine, diclofenac, ketorolac, flurbiprofen, suprofen, idoxuridine, trifluridine, cidofovir acyclovir, famciclovir, valacvclovir, cromolyn sodium, ketorolac tromethamine, levocabastine ketotifen, iodoxamide, emedastine, olopatadine, loteprednol etabonate, pemerolast potassium, levofloxacin, amphotericin B, nystatin, miconazole, and ketoconazole.

16. The applicator of claim 14 wherein the medication is an antifungal.

17. The applicator of claim 14 wherein the medication is an antibiotic.

18. The applicator of claim 14 wherein the medication is a mast cell stabilizer.

19. The applicator of claim 14 wherein the medication is a corticosteroid.

20. The applicator of claim 14 wherein the applicator is used while the person has an earplug in his ear canal.
